(19) United States
(12) Patent Application Publication
(10) Pub. No.: US 2008/0306126 A1
(43) Pub. Date: Dec. 11, 2008

(54) Peroxisome proliferator activated receptor treatment of hyperhomocysteinemia and its complications

(76) Inventors: Vivian A. Fonseca, New Orleans, LA (US); Dennis B. McNamara, New Orleans, LA (US)

Correspondence Address: Vivian A. Fonseca, 1430 Tulane Avenue SL53, New Orleans, LA, 70112, US

(21) Appl. No.: 11/029,048

(22) Filed: Jan. 4, 2005

Related U.S. Application Data
(60) Provisional application No. 60/534,285, filed on Jan. 5, 2004.

(51) Int. Cl. A61K 31/426

(52) U.S. Cl. 514/369

(57) ABSTRACT

A method of lowering homocysteine level of an individual and of reducing the vascular pathology associated therewith, by administering a ligand of the peroxisome proliferator activate receptor gamma. The peroxisome proliferator activated receptor preferably may be rosiglitazone.

### STATEMENT REGARDING FEDERALLY SPONSORED RESEARCH

Not Applicable

### CROSS-REFERENCE TO RELATED APPLICATIONS

This application claims the benefit of U.S. Provisional Patent Application No. 60/534,285, filed Jan. 5, 2004.

### FIELD OF THE INVENTION

The current invention relates to pharmaceutical preparations to reduce homocysteine levels of individuals.

### BACKGROUND

Homocysteine is an amino acid and an intermediate formed during the metabolism of the essential amino acid, methionine to cysteine. It is either totally absent or present in very small quantities in various foods. Plasma homocysteine, however is derived from methionine, and its concentration in blood can increase due to defective metabolism, usually related to defects in the activity of one of several enzymes involved in its metabolism. The metabolic alterations could either be i) acquired, as are the co-factor (vitamin) deficiencies, or ii) due to mutations in genes coding for the enzymes of metabolism of homocysteine (1-3). Inborn errors of metabolism and related disorders are well documented in literature (4, 5). Hyperhomocysteinemia, or increased serum concentrations of homocysteine, is generally recognized as an independent risk factor for coronary, cerebral, and peripheral atherosclerosis (6-8). Environmental influences, medications, physiological status such as disease, pregnancy and lactation could contribute to variations in the levels of homocysteine (9-12). The circulating levels of homocysteine are also contributed by cellular export mechanism, and these are known to increase in various conditions (13). Transitory elevations in methionine may occur following meals rich in this amino acid. This increase in methionine may lead to an increase in homocysteine levels. The effect of this transitory elevation in methionine/homocytseine levels on the development of vascular or other pathology, as detailed below, is uncertain.

Hyperhomocysteinemia is associated with vascular disease in general, particularly in subjects with significant carotid stenosis (14-16). Clinical and epidemiological studies have shown that homocysteine measured in serum or plasma is a strong predictor of cardiovascular disease risk (17). Hyperhomocysteinemia can be moderate (16-30 μmoles/L), medium (30-100 μmoles/L) or severe (>100 μmoles/L (18). Mild to severe hyperhomocysteinemia has been reported to cause pathologic changes in the vascular wall, neural tube formation and kidney function. Previous workers have shown that plasma total homocysteine level is a strong predictor of mortality in patients with angiographically confirmed coronary artery disease (16). Elevated serum homocysteine is associated with sudden unexpected death and is especially associated with diabetes (19). In addition, elevated serum homocysteine is associated with an increase in fibrous plaques and a relative decrease in thin-cap atheromas (19). It is also reported that in the absence of other known risk factors hyperhomocysteinemia stimulates the expression of adhesion molecules such as MCP-1, VCAM-1, and E-selectin in vivo, leading to increased monocyte adhesion to the aortic endothelium, which may significantly contribute to the development of atherosclerosis by facilitating monocyte/macrophagre infiltration into the arterial wall (20).

Current treatments for homocysteine include medications that consist of vitamin supplements containing a variety of B vitamins. These treatments have been shown to reduce the homocysteine levels of animals fed methionine-enriched diets (21). However, the benefits of these vitamin therapies tend to be very general. It is still not clear which of the vitamins in these mixtures offer homocysteine-lowering benefits. Because of the association between homocysteine and cardiovascular disease, there exists a need for a more robust pharmaceutical treatment.

The inventors have shown that a subtype of peroxisome proliferator activated receptors (PPAR) are capable of lowering serum homocysteine levels in individuals. The peroxisome proliferator activated receptors are ligand-activated transcription factors that increase transcription of target genes by binding to a specific nucleotide sequence in the gene's promoter. These serve as receptors for two very important classes of compounds: hypolipidemic drugs (fibrates, agonists of PPARα) and the insulin sensitizing drugs (thiazolidinediones, agonists of PPARγ). The beneficial effects of PPARγ ligands include improving insulin sensitivity, decreasing of hyperinsulinemia, increasing HDL, lowering blood pressure, decreases in the generation of reactive oxygen species, and improving vascular reactivity. The PPARγ ligand, rosiglitazone has been shown to have protective effects on the vessel wall (22-24).

The inventors have shown that the augmentation of the vascular response to injury by hyperhomocystenemia is attenuated by the PPARγ agonist rosiglitazone. In addition, the inventors have shown that a PPARγ agonist can decrease a methionine diet-induced hyperhomocysteinenia. The intimal hyperplasia that developed in the methionine diet group in response to the catheter injury was 4-fold higher than that in the control diet group (I/M=0.2+/−0.04 vs. 0.82+/−0.21, respectively). Rosiglitazone treatment reduced the homocysteine level to 16.4+/−1.7 uM and reduced the I/M to 0.27+/−0.12. The reduction in the intimal hyperplasia in response to rosiglitazone was not significantly different from the group on the control diet; however, this reduction in intimal hyperplasia was associated with a homocysteine level, that while significantly reduced, remained significantly elevated as compared with the control diet group (p=0.001) Thus the intimal hyperplastic response exhibited a dose responsive relationship with respect to the plasma homocysteine. Moreover, a normal hyperplastic response to catheter injury was observed in the presence of significantly elevated homocysteine levels. Thus, the inventors have shown that rosiglitazone exerts its effect on intimal hyperplasia through a mechanism other than, or in addition to, a reduction in plasma homocysteine levels. This finding supports the suggestion that some product or products of excess methionine metabolism, rather than high homocysteine only may contribute to the association of high homocysteine with vascular disease (25). The findings of the inventors suggest if a product of excess methionine metabolism other than high plasma homocysteine underlies the association of homocysteine with vascular disease that rosiglitazone, and perhaps PPARγ ligands as a class, suppress the vascular pathology associated with this/these presently unknown metabolite(s) of methionine.

The inventors have shown that with respect to the reduction in plasma homocysteine levels, the present study demonstrates that rosiglitazone stimulated the activity of CβS. This observation confirms and extends our earlier report that troglitazone stimulated CβS activity in the fatty Zucker rat (26).

Another mechanism by which intimal hyperplasia can be reduced is inhibition of VSMC proliferation. The inventors' data confirm that of previous studies in that exposure of VSMCs to high levels of homocysteine (100 uM) resulted in increased cell proliferation (FIG. 5). However, they show that rosiglitazone completely inhibited the proliferative response to homocysteine. The VSMC proliferation data are paralleled and confirmed by the inhibition of DNA synthesis (FIG. 6). Homocysteine stimulated 3-thymidine incorporation in VSMCs. Rosiglitazone completely inhibited homocysteine-stimulated DNA synthesis. Rosiglitazone inhibited 3-thymidine incorporation in control cell cultures. The inventors show that in addition to increasing homocysteine metabolism via CβS, that rosiglitazone inhibits the development of intimal hyperplasia directly by inhibiting VSMC proliferation and DNA synthesis.

### SUMMARY OF THE INVENTION

The inventors have developed a method of lowering the serum homocysteine levels of an individual. Also provided is a method of lowering the homocysteine levels of an individual suffering from diet induced intimal hyperplasia.

In another embodiment of the present invention, methods of reducing the homocysteine levels of an individual are provided. More specifically, the present invention provides methods of reducing homocysteine levels in an individual by the use of compounds of the thiozolidinedione class. Also provided are methods of preventing the development of diet-induced increases in homocysteine using compounds of the thiozolidinedione class. More specifically, the present invention provides methods of reducing homocysteine levels in an individual by the use of the thiozolidinediones rosiglitazone. Also provided are methods of preventing the development of diet-induced increases in homocysteine using the thiozolidinedione rosiglitazone.

The present invention also provides methods for reducing vascular pathology which is produced by elevated levels of homocysteine.

### BRIEF DESCRIPTION OF THE DRAWINGS

FIG. 1. Representative micrographs of balloon catheter-injured carotid arteries from fatty Zucker rats on various diets: A, control diet; B, methionine diet; C, control diet with rosiglitizone; and, D, methionine diet with rosiglitazone.

FIG. 2. Summary data for the effect of rosiglitazone on the rate of development of intimal hyperplasia. The “n” for each group was: control diet, 7; control diet with rosiglitazone, 7; methionine diet, 6; and, methionine diet with rosiglitazone, 8.

FIG. 3. Summary data for homocysteine levels: the effect of rosiglitazone. The “n” for each group was: control diet,; control diet with rosiglitazone,; methionine diet,; and methionine diet with rosiglitazone.

FIG. 4. Cystathionine β synthase activity: the effect of rosiglitazone. The “n” for each group was 5.

FIG. 5. VSMC Proliferation: the effect of rosiglitazone. The “n” for each group was 24.

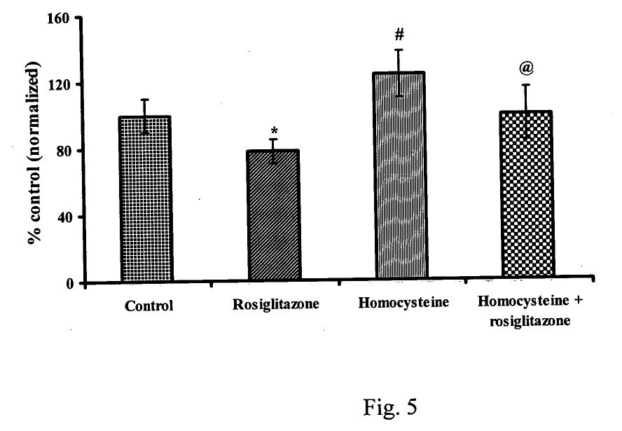

FIG. 6. DNA (3-thymidine) synthesis: the effect of rosiglitazone. The “n” for each group was 3.

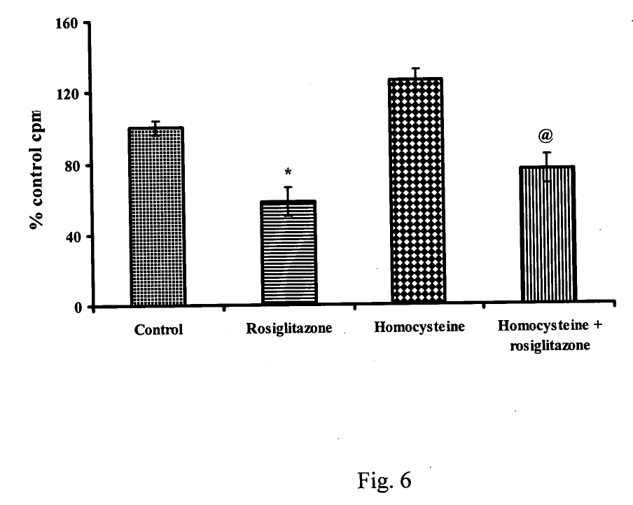

### DETAILED DESCRIPTION OF THE INVENTION

Ten-week-old female Sprague Dawley rats were obtained from Harlan Laboratories (Indianapolis, Ind.). After an acclimatization period of one week, they were fed a powdered control diet with or without methionine for one week. After one week on the normal or methionine diet, an aqueous preparation rosiglitazone (3.0 mg/kg/d) or vehicle was administered by gavage. The methionine diet was prepared with 10 g of L-methionine obtained from Sigma Chemical Company in St. Louis, Miss., added to 10 kg of powdered feed and thoroughly mixed. The rats were divided into four groups (n=7/group) and fed a) control diet; b) control diet with administration of rosiglitazone; c) methionine diet; and d) methionine diet with rosiglitazone.

Balloon catheter injury of to the carotid artery was induced as previously published (27). Briefly, a 2.0 mm PTCA obtained from Boston Scientific, Inc. in Quincy, Mass. was introduced through the femoral artery to the left carotid. The balloon was inflated to 4 atmospheres for 20 seconds and then deflated to 2 atmospheres and dragged down to the aorta. The animals were continued on their respective diets and rosiglitazone treatment was continued as indicated for three weeks after the surgery. The surgical procedures were conducted under anesthesia in a totally aseptic atmosphere. All rats had free access to the feed and water, and were maintained in a 12 hour day/light cycle.

Before the beginning of the experiment, and at the time of sacrificing the animal a blood sample was obtained from each rat. At the end of the experiment, rats were sacrificed using a carbon dioxide chamber for harvesting tissues. Samples of blood and liver were collected and immediately processed. The serum and liver samples were stored frozen.

The Hcy levels were measured by high pressure liquid chromatography according to an established method (28,29). Cystathionine-β-synthase (CβS) was assayed in the liver samples, and the procedure followed was as previously published (26).

The carotid arteries were separated and flushed with zinc buffered formalin. Each artery was cut from top into 4 equal segments and placed sequentially for embedding in paraffin. Sections no thicker then 6 um were cut and stained with hemotoxalin and eosin for microscopy. All four segments were read and the intimal/medial (I/M) ratio was calculated using Image-Pro software (NIH). The mean of the I/M ratios of the 4 sections reflected the I/M for one animal.

### Histology

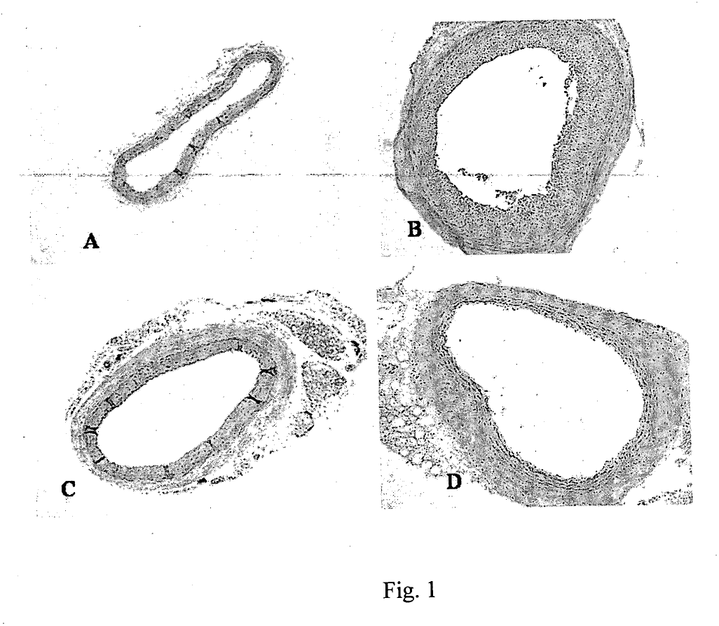

Representative micrographs of sections of the carotid artery of Sprague Dawley rats sacrificed 21 days following the balloon catheter-induced injury are presented in FIG. 1. Micrograph A is from an animal that was fed the control diet. As can be observed, development of intimal hyperplasia occurred over the three-week interval following catheter injury; the IM ratio was 0.13. There was not a significant change in the medial area following catheter injury in any of the animals. Therefore, a change in I/M represents a change in the intimal area. Micrograph B is from a carotid artery of an animal on the methionine diet; the I/M was 1.37. Micrograph C is from an animal that was on the control diet and also administered rosiglitazone; the I/M ratio was 0.19. Micrograph D is from an animal that received the methionine diet as well as rosiglitazone; the I/M ratio was 0.17. Thus the animal on the methionine diet exhibited augmented development of intimal hyperplasia and this augmentation was attenuated by treatment with rosiglitazone.

### Development of Intimal Hyperplasia

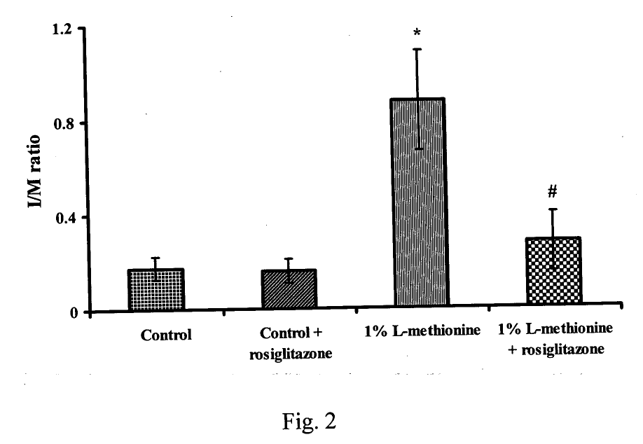

Summary data for the effect of rosiglitzone on the rate of development of intimal hyperplasia are presented in FIG. 2. The group of animals received the control diet had an I/M of 0.18+/−0.05; the group on the control diet that received rosiglitazone exhibited an I/M of 0.21+/−0.03; this was not statistically significant, as compared to the control diet group without rosiglitazone, (p=0.997). However, in the group that received the methionine diet, the development of intimal hyperplasia was markedly augmented; this group exhibited an I/M of 0.82+/−0.21 which was significant, as compared to the control diet group (p=0.005). Administration of rosiglitazone to the group receiving the methionine diet inhibited the development of intimal hyperplasia seen in the presence of the methionine diet alone, I/M of 0.28+/−0.12, (p=0.018). Moreover, the development of intimal hyperplasia in the group with methionine diet plus rosiglitazone treatment was not statistically different from that seen in the group on the control diet without rosiglitazone (0.28+/−0.12 vs. 0.18+/−0.05, respectively (p=0.918). The I/M in animals that did not undergo balloon catheter-induced injury was zero.

### Homocysteine Levels

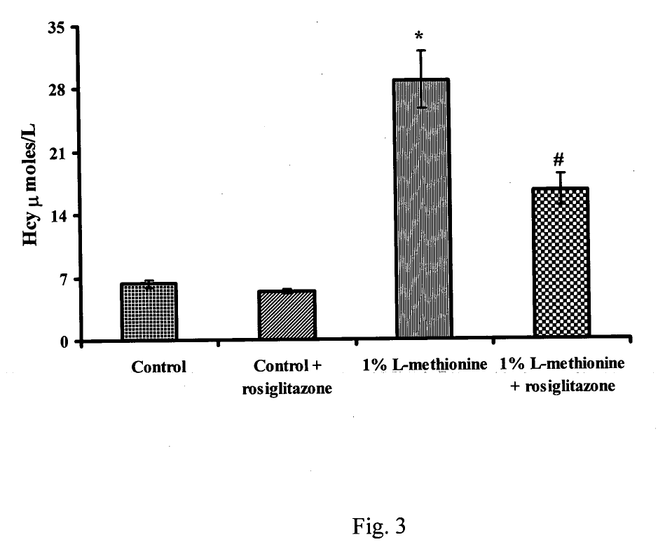

Summary data for serum homocysteine levels are present in FIG. 3. Homocysteine levels in animals on the control diet were 6.3+/−0.04 μM. Administration of rosiglitazone to animals on the control diet did not affect the homocysteine levels, 5.1+/−0.6 μM. The group receiving the methionine diet exhibited markedly enhanced levels of homocysteine, 28.9+/−3.2 μM. This increase was statistically significant as compared with the control diet (p=0.002). Administration of rosiglitazone to the animals on the methionine diet significantly reduced the serum homocysteine concentration, 16.4+/−1.7 μM. This effect was statistically significant as compared to the methionine diet alone (p=0.001); however, the homocysteine levels remained statistically elevated as compared to the control diet group, (p=0.002). Catheter injury did not affect plasma homocysteine levels in animals on the control diet.

### CβS Activity

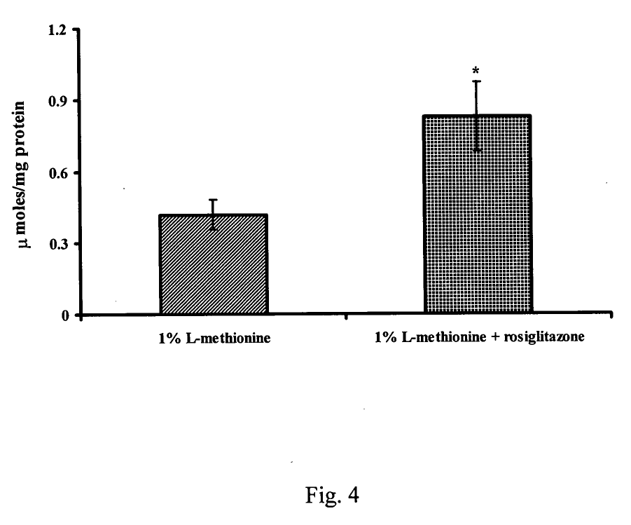

The enzyme that catabolizes homocysteine converting it to cystathionine is cystathionine-β-synthase (CβS). The activity of this enzyme in the animals receiving the methionine diet was 0.35+/−0.05 μmoles/mg protein (FIG. 4). Rosiglitazone augmented the activity of CβS in the animal on the methionine diet, 0.57+/−0.1 μmoles/mg protein.

### REFERENCES

1. Stabler S. P. Vitamins, homocysteine, and cognition. Am J Clin Nutr. 78: 359-60, 2003.
2. Tsai M. Y., Hanson N Q, Bignell M K, Schwichtenberg K A. Simultaneous detection and screening of T833C and G919A mutations of the cystathionine beta-synthase gene by single-strand conformational polymorphism. Clin Biochem 5: 473-7, 1996.
3. Rothenbacher D., Fischer H. G, Hoffmeister A, Hoffmann M M, Marz W, Bode G, Rosenthal J, Koenig W, Brenner H. Homocysteine and methylenetetrahydrofolate reductase genotype: association with risk of coronary heart disease and relation to inflammatory, hemostatic, and lipid parameters. Atherosclerosis. 162:193-200, 2002.
4. Scriver C. R., Baudet, A. L., Sly, W. S., Valle, D. (eds): The metabolic bases of inherited disease, 8th ed. McGraw Hill Inc. New York, 2001.
5. King R. A., 2002 Rotter, J. I., Motulsky, A. G. (eds): The genetic basis of common diseases, 2nd ed.; Oxford University Press, Inc., New York, 2002.
6. McCully, K. S. Vascular Pathology of homocystenemia-Implication for the pathogenesis of arterioslerosis. Am. J. Pathol.; 56 (1): 111-28, 1969.
7. McCully, K. S, Wilson, R. Homocysteine theory of atherosclerosis. Atherosclerosis. 22 (2): 215-227, 1975.
8. McCully K. S. Chemical pathology of homocysteine. I. Atherogenesis. Ann Clin Lab Sci. 24(1):27-59 1994.
9. Desouza, C., Keebler, M., McNamara, D. B., Fonseca, V. Drugs affecting homocysteine metabolism: impact on cardiovascular risk. Drugs. 62:605-16, 2002.
10. Sacco R. L. Newer risk factors for stroke. Neurology. 57: S31-4, 2001.
11. Cotter A. M., Molloy, A. M., Scott, J. M., Daly, S. F. Elevated plasma homocysteine in early pregnancy: a risk factor for the development of nonsevere preeclampsia. Am J Obstet Gynecol. 189(2):391-4, 2003.
12. Ramlau-Hansen, C. H., Moller, U. K., Moller, J., Thulstrup, A. M. Lactation-a risk factor for elevated plasma homocysteine? Ugeskr Laeger. 165:2819-23, 2003.
13. Hultberg B. Modulation of extra cellular homocysteine concentration in human cell lines. Clin Chim Acta. 330:151-9, 2003.
14. Selhub, J., Jacques, P. F., Bostom, A. G., D'Agostino, R. B., Wilson, P. W., Belanger, A. J., O'Leary, D. H., Wolf, P. A., Schaefer, E. J., Rosenberg, I. H. Association between plasma homocysteine concentrations and extracranial carotid-artery stenosis. N Engl J Med. 332:286-91, 1995.
15. Streifler J. Y., Rosenberg, N., Chetrit, A., Eskaraev, R., Sela, B. A., Dardik, R., Zivelin, A., Ravid, B., Davidson, J., Seligsohn, U., Inbal, A. Cerebrovascular events in patients with significant stenosis of the carotid artery are associated with hyperhomocysteinemia and platelet antigen-1 (Leu33Pro) polymorphism. Stroke. 32: 2753-8, 2001.
16. Nygard, O., Nordrehaug, J. E., Refsum, H., Ueland, P. M., Farstad, M., Vollset, S. E. Plasma homocysteine levels and mortality in patients with coronary artery disease. N. Engl. J. Med. 337: 230-6, 1997.
17. Ueland, P. M. and Resfsum, H. Plasma homocysteine, a risk factor for vascular disease: plasma levels in health, disease, and drug therapy. J. Lab Clin. Med. 114: 473-501, 1989.
18. Korczyn, A. D. Homocysteine, Stroke and Dementia. Stoke. 33: 2343-2344, 2002.
19. Burke, A. P., Fonseca, V., Kolodgie, F., Zieske, A., Fink, L., Virmani, R. Increased Serum Homocysteine and Sudden Death Resulting from Coronary Atherosclerosis With Fibrous Plaques. Arteriosclerosis, Thrombosis & Vascular Biology. 22: 1936-1941, 2002.
20. Wang, G., Woo, C. W., Sung, F. L., Siow Y. L., Karmin, O. Increased Monocyte Adhesion to Aortic Endothelium in Rats With Hyperhomocysteinemia: Role of Chemokine and Adhesion Molecules. Arterioscler. Thromb. Vasc. Biol. 22 (11): 1777-1783, 2002.
21. Narin F; Narin N; Akcakus M; Ustdal M; Karakucuk I; Halici C. The effect of folic acid, vitamin B6 and vitamin B12 on the homocysteine levels in rabbits fed by methionine-enriched diets. The Tohoku journal of experimental medicine, 198(2):99-105
22. Desouza, C. V., Murthy, S. N., Diez, J., Dunne, B., Matta, A. S., Fonseca, V. A., McNamara, D. B. Differential effects of peroxisome proliferators activator receptor α and γ ligands on intimal hyperplasia after balloon catheter induced vascular injury in Zucker rats. J Cardiovascular Pharmacology & Therapeutics 8(4): 297-306, 2003.
23. Choi, S. H., Choi, D. H., Ko, Y. K., et al. Preventive effects of rosiglitazone on restenosis after coronary stenting in patients with type-2 diabetes (Abstract). Diabetes. 52 Suppl. 1): A19. Abstract 82-OR, 2003.
24. Yamakawa, K., Hosoi, M., Koyama, H., Tanaka, S., Fukumoto, S., Morii, H., Nishizawa, Y. Peroxisome proliferator-activated receptor-gamma agonists increase vascular endothelial growth factor expression in human vascular smooth muscle cells. Biochem Biophys Res Commun. 271:571-574, 2000.
25. Troen, A., Lutgens, E., Smith, D., Rosenberg, I., Selhub, J. The atherogenic effect of excess methionine intake. PNAS 100: 15089-15094, 2003.
26. Fonseca, V., Keebler, M., Dicker-Brown, A., DeSouza, C., Poirer, L. A. Murthy, S. N. McNamara, D. B. The effect of troglitazone on plasma homocysteine, hepatic and red cell S-adneosyl methionine and S-adenosyl homocysteine and enzymes in homocysteine metabolism in Zucker rats. Metabolism 51:783-6, 2002. Uerre, J. A., Miller, C. H. Preparation of L-homocysteine from L-homocysteine thiolactone. Anal. Biochem 17: 310-5, 1996.
27. Schiller, N. K., McNamara, D. B. Balloon catheter vascular injury of the alloxan-induced diabetic rabbit: the role of-insulin-like growth factor-1. Mol Cell Biochem. 202(1-2):159-167, 1999.
28. Toyo'oka, T., Suzuki, T., Saito, Y., Uzu, S., Imai, K. Fluorogenic reagent for thiols: 4-(N,N-dimethylaminosulphonyl)-7-fluoro-2,1,3-benzoxadiazole. Analyst. (1989) 114(4):413-9.
29. Murthy. S., Matta, S., Mondal, D., McNamara, D. Methods in assessing homocysteine metabolism. Metabolic Syndrome and Related Disorders Vol. 1, pp 129-140, (Mary Ann Liebert, Inc.), 2003.

We claim:

1. A method of lowering the homocysteine level of an individual by administering a ligand of the peroxisome proliferator activated receptor gamma.

2. A method of reducing the vascular pathology associated with elevated homocysteine levels by administering a ligand of the peroxisome proliferator activated receptor gamma.

3. A method of claim 1, wherein said ligand of the peroxisome proliferator activated receptor gamma is rosiglitazone.

4. A method of claim 2, wherein said ligand of the peroxisome proliferator activated receptor gamma is rosiglitazone.
